(19) United States
(12) Patent Application Publication
(10) Pub. No.: US 2006/0121040 A1
(43) Pub. Date: Jun. 8, 2006

(54) Compositions and methods for treating neuroendocrine tumors

(75) Inventors: Muthusamy Kunnimalaiyaan, Madison, WI (US); Herbert Chen, Madison, WI (US)

Correspondence Address: CROWELL & MORING LLP;INTELLECTUAL PROPERTY GROUP, P.O. BOX 14300, WASHINGTON, DC, 20044-4300, US

(73) Assignee: Wisconsin Alumni Research Foundation, Madison, WI (US)

(21) Appl. No.: 11/296,404

(22) Filed: Dec. 8, 2005

Related U.S. Application Data
(60) Provisional application No. 60/634,082, filed on Dec. 8, 2004.

(51) Int. Cl. A61K 48/00; A61K 39/395; A61K 31/5513; A61K 31/407; A61K 31/381

(52) U.S. Cl. 424/146.1; 514/44; 514/221; 514/410; 514/438

(57) ABSTRACT

Methods and pharmaceutical compostions for treating or suppressing symptoms of neuroendocrine (NE) tumors comprising inhibiting the activities of GSK-3β of the cancer cells. Also disclosed are pharmaceutical compositions for the methods. Preferably, the pharmaceutical composition comprises Li+, SB216763, SB415286, indirubins, Paullones, Hymenialdisine, Azakenpaullone, Thienyl and phenyl α-halomethyl ketones, CHR 99021, AR-A014418, Bis-7-azaindolylmaleimides, CHR 98023, CHR-98014, and ZM336372, or a pharmaceutically acceptable salt or derivative thereof.

### CROSS-REFERENCE TO RELATED APPLICATION

This application claims the benefit of the filing date of Provisional Application Ser. No. 60/634,082, filed Dec. 8, 2004, the entire disclosure of which is hereby incorporated by reference.

### GOVERNMENT INTEREST

This invention was made with United States government support awarded by the National Institutes of Health under the grant number NIH DK064735. The United States has certain rights in this invention.

### FIELD OF THE INVENTION

This invention relates to compositions and method for treating neuroendocrine tumors by reducing the activity of glycogen synthase kinase 3 (GSK3).

### BACKGROUND OF THE INVENTION

Neuroendocrine (NE) tumors such as carcinoid and islet cell tumors frequently metastasize to the liver, and are second only to colorectal carcinoma as the most common source of isolated hepatic metastases (Siperstein et al., 1990, World J. Surg. 25:693-696; Chen et al., 1998, J. Gastrointest. Surg. 2:151-155; Elias et al., 1998, J. Am. Coll. Surg. 187:487-493). Over 90% of patients with pancreatic carcinoid tumors and 50% of patients with islet cell tumors develop isolated hepatic metastases (Creutzfeldt W., 1996, World J. Surg. 20:126-131; Hiller et al., 1998, Abdom. Imaging 23:188-190; Mavligit et al., 1993, Cancer 72:375-380; Kebebew et al., 2000, Arch. Surg. 135:895-899; Isozaki et al., 1999, Intern. Med. 38:17-21). Patients with untreated, isolated NE liver metastases have less than 30% 5-year survival probability (Siperstein et al., 1990, World J. Surg. 25:693-696; Elias et al., 1998, J. Am. Coll. Surg. 187:487-493). While surgical resection can be potentially curative, 90% of patients are not candidates for hepatectomy due to the degree of hepatic involvement by NE tumors (Nave et al., 2001, Surgery 129(2):170-175170-1755).

Besides surgery, however, there are no curative treatments for NE tumors and their hepatic metastases. Presently available alternatives to surgery, including chemoembolization, radiofrequency ablation, cryoablation, chemotherapy, and liver transplantation, have limited efficacy (Brown et al., 1999, J. Vasc. Interv. Radiol. 10:397-403; Isozaki et al., 1999, Intern. Med. 38:17-21; Miller et al., 1998, Oncol. Clin. N. Am. 7:863-879; Prvulovich et al., 1998, J. Nucl. Med. 39:1743-1745; Eriksson et al., 1998, Cancer 83:2293-2301; Lehnert T., 1998, Transplantation 66:1307-1312; Zhang et al., 1999, Endocrinology 140:2152-2158).

In general, chemotherapy has had limited success in patients with NE tumors. Multiple chemotherapeutic agents have been assessed alone or in combination for patients with advanced neuroendocrine tumors. The response rate to chemotherapy in metastatic carcinoid tumors has been reported to be no higher than about 20-30%. In endocrine pancreatic tumors, streptozocin combined with doxorubicin has been reported to generate responses in 69% of patients; however, the determination of response in this trial contained methods unacceptable to today's standards (Sippel and Chen, Problems in General Surgery 2004; 20:125-133). Of note, researchers at the Memorial Sloan-Kettering Cancer Center (MSKCC) reported a patient series treated with this regimen with a response rate of only 6% as determined by standard clinical trial criteria. The chemotherapeutic regimens recommended in neuroendocrine tumors are associated with significant toxicities. For example, the toxicities associated with streptozocin/doxorubicin include vomiting (80% of all patients, 20% severe vomiting), leukopenia (57%), renal insufficiency (44%), stomatitis, and diarrhea. Clearly in patients with a potentially indolent disease, reducing the toxicities associated with treatment is of utmost importance. Less toxic, effective therapies for this population of patients are urgently needed.

Furthermore, patients with liver metastases from NE tumors often have debilitating symptoms, such as uncontrollable diarrhea, flushing, skin rashes, and heart failure due to the excessive hormone secretion that characterizes these tumors. Thus, patients with incurable disease frequently have a poor quality of life. Therefore, for the majority of patients with NE and their hepatic metastases, there is a need for the development of other forms of therapy.

### SUMMARY OF THE INVENTION

The invention generally provides methods for treating neuroendocrine (NE) tumors by inhibiting the expression level or activity of glycogen synthase kinase 3 (GSK3), for example by administering to a patient in need thereof a pharmaceutical composition comprising an effective amount of a GSK inhibitor or antagonist.

GSK3 is known to be an essential component of the mechanism that determines cell-fate and is involved in the regulation of protein syntheses, cell proliferation, microtubule assembly and disassembly, and apoptosis.

The present inventors have determined that the activation or up-regulation of raf-1 and various factors involved in the raf-1 signaling pathway are detrimental to NE cancer cells (Sippel et al., Am J Physiol Gastrointest Liver Physiol 2003; 285:G245-G254; Chen et al., Surgery 1996; 120:168-172), including suppression of neuroendocrine marker and hormone levels in human gastrointestinal carcinoid cells, induction of cancer cell differentiation, and silencing of expression of the neural transcription factor human achaete-scute homolog-1 (hASH-1).

In ras/raf-1 signaling pathway, extracellular signals transmitted through growth factor receptors lead to activation of ras. Activated ras then translocates raf-1 to the cell membrane allowing phosphorylation of MEK and MAP kinases. These events lead to activation of transcription factors that control cell growth and differentiation. The role of the various isoforms of ras (K-ras, H-ras, and N-ras) in neuroendocrine (NE) tumors has been shown to differ significantly from other malignancies. In non-NE tumors, ras activating mutations are quite prevalent, occurring in 30% of all human cancers (Bos, Cancer Res 1989; 49:4682-4689). In contrast, NE tumors rarely have detectable ras mutations. Of almost 100 gastrointestinal (GI) NE tumors (carcinoids, insulinomas, gastrinomas, and glucagonomas) characterized, no ras mutations have been found (Younés et al., Cancer 1997; 79:1804-1808). In studies of NE lung tumors, fewer than 1% of pulmonary carcinoids and small cell lung cancers had ras mutations (Onuki et al., Cancer 1999; 85:600-607).

As indicated above, medullary thyroid cancer cells have been shown to respond to a ras signal with a differentiation response (Nakagawa et al., Proc Natl Acad Sci USA 1987; 84:5923-5927), and this has been confirmed with an in vitro model of medullary thyroid cancer tumor differentiation using an inducible raf-1 construct (Chen et al., Surgery 1996; 120:168-172). In this system, activation of raf-1 in medullary thyroid cancer cells causes cessation of growth, phenotypic differentiation, and downregulation of the RET proto-oncogene (Carson-Walter et al., Oncogene 1998; 17:367-376). Similarly, in pheochromocytoma cell lines, induction of ras also results in cessation of cell growth (Wood et al., Proc Natl Acad Sci USA 1993; 90:5016-5020). In addition, ras/raf-1 activation in small cell lung cancer cells results in suppression of growth capacity, loss of soft agar cloning ability, and cell cycle arrest (Ravi et al., Am J Respir Cell Mol Biol 1999; 20:543-549).

Glycogen synthase kinase-3 (GSK3) is a proline-directed serine-threonine kinase that was initially identified as a phosphorylating and inactivating glycogen synthase. Two isoforms, alpha (GSK3A) and beta, show a high degree of amino acid homology. GSK3B, or GSK3β was first described in a metabolic pathway for glycogen synthase regulation (Cohen et al., Biochem. Soc. Symp. 1978; 43:69-95). It is now clear that GSK3B is a multifunctional kinase that regulates numerous cellular processes, such as metabolism, cell fate determination, proliferation, and survival (Hardt & Sadoshima, Circulation Research 2002; 90:1055-1063; Krylova et al., Journal of Cell Biology 2000; 151:83-93; Harwood et al., Cell 1995; 80:139-148; and Wang et al., J. Biol. Chem. 2002; 277:36602-36610). GSK3B has a wide range of substrates such as B-catenin, c-myc, c-Jun, and heat shock factor (Aberle et al., EMBO J. 1997; 16:3797-3804; Beals et al., Science 1997; 275:1930-1933; Degroot et al., Oncogene 1993; 8:841-847; He et al., Molecular and Cellular Biology 1998; 18:6624-6633; Rogatsky et al., J. Biol. Chem. 1998; 273:14315-14321; and Sears et al.,. Genes & Development 2000; 14:2501-2514). The activity of GSK3B is controlled by phosphorylation; GSK3B becomes inhibited by phosphorylation of a single serine residue (Ser9). Thus, in contrast to other kinases, GSK3B is non-phosphorylated and highly active in unstimulated cells, and becomes inactivated (phosphorylated) in response to signaling cascades including the raf-1/MEK/ERK1/2 pathway (Cohen and Frame, Nature Reviews Molecular Cell Biology 2001; 2:769-776).

The present inventors discovered that that inhibition of GSK-3β leads to the treatment or reduction in symptoms of NE tumors; specifically, GSK-3β inhibitors suppress NE tumor proliferation and hormone production. Accordingly, the present invention provides a method for treating neuroendocrine (NE) tumors, or for inhibiting or reducing symptoms of NE tumors in a patient, comprising administering to the patient a therapeutically effective amount of a pharmaceutical composition which comprises a pharmaceutically acceptable amount of a GSK-3 specific inhibitor that is sufficient to block or inhibit activity of GSK-3 in the patient. Preferably, GSK3β inhibitors or antagonists are used, such as Li+, SB216763, SB415286, indirubins, Paullones, Hymenialdisine, Azakenpaullone, Thienyl and phenyl α-halomethyl ketones, CHR 99021, AR-A014418, Bis-7-azaindolylmaleimides, CHR 98023, CHR-98014, and ZM336372, or a pharmaceutically acceptable salt or derivative thereof.

In a preferred embodiment, the GSK3 inhibitors suitable for the present invention include anti-GSK-3 antibody, or a polynucleotide molecule comprising a sequence that is antisense to a nucleic acid encoding a GSK-3, or a small interfering RNA (siRNA) based on a nucleic acid encoding a GSK-3. Preferably, the siRNA is based on a genomic sequence encoding GSK-3β, or it may be based on a cDNA sequence encoding GSK3β.

According to the present invention, blocking or inhibiting the GSK-3 activity in the patient results in reducing formation of chromogranin A (CgA) or human achaete-scute homolog-1 (hASH1) in NE tumor cells.

NE tumors suitable for treatment according to the method of the present inveinto include but are not limited to carcinoids, islet cell tumors, and medullary thyroid cancers.

The present invention further provides a pharmaceutical composition for treating a neuroendocrine (NE) tumors, or for inhibiting or reducing symptoms of NE tumors in a patient, the pharmaceutical composition comprising a therapeutically effective amount of a pharmaceutically acceptable amount of a GSK-3 specific inhibitor that is sufficient to block or inhibit activity of GSK-3 in the patient, and a pharmaceutically acceptable excipient. The present invention, in another embodiment, provides a kit that comprises the pharmaceutical composition of the present invention, and an instructional material regarding the use thereof to treat an NE tumor or reducing a symptom thereof, and optionally a delivery device for delivering the composition to a patient.

Also provided are kits for administering an effective amount of an inhibitor of GSK-3 activity, such as lithium, in a subject having a NE tumor.

### BRIEF DESCRIPTION OF THE DRAWINGS

Other objects, advantages and novel features of the present invention will become apparent from the following detailed description when considered in conjunction with the accompanying drawings.

FIG. 1 shows that treatment of human medullary thyroid cancer cells with increasing doses of lithium chloride (0-100 mM) led to an increase of phosphorylated GSK3 level, and a reduction, in a dose responsive manner, of neuroendocrine (NE) cancer markers, chormogranin A (CgA) and human achaete-scute homolog-1 (hASH1).

FIG. 2 shows that treatment of medullary thyroid cancer TT and carcinoid H727 cells with lithium chloride (LiCl, 20 mM) inhibited cellular growth (solid lines) compared to control cells (C) (dotted lines).

FIG. 3A shows that lithium chloride inhibits growth of carcinoid tumor cells after only 4 doses. FIG. 3B shows the effect of lithium on in vivo NE tumor growth. Animals with established NE tumors were treated with control or lithium (400 μg/kg) for 2 weeks.

FIG. 4 shows that treatment of animals with varying doses of lithium chloride every 2 days for 10 treatments significantly reduces tumor sizes. (A) control; (B) 0.5M; (C) 2M.

FIG. 5A shows that lithium reduces levels of chromogranin A in murine carcinoid tumors; FIG. 5B shows similar results for phosphorylated GSK3B and chromogranin A on a human GI NE tumor sample.

FIG. 6 shows that treatment of human medullary thyroid cancer cells with SB216763 led to reduction of CgA and hASH1.

FIG. 7 shows that treatment of NE tumor cells with ZM336372 (0-50 μM) led to increased levels of phosphorylated GSK3B and ERK1/2.

FIG. 8 shows that activation of Raf-1 by estradiol in BON-raf cells leads to the phosphorylation of MEK 1/2 and ERK 1/2 proteins and reduces the level of chromogranin A (CgA) and hASH1 proteins significantly in a timedependent manner. Western analysis for Raf-1 pathway activation and its downstream effect. Total cellular extracts from BON and BON-raf cells treated with carrier [(C), ethanol] and estradiol (E2) for indicated days and analyzed against various antibodies as shown. G3PDH was used as loading control. There is no reduction of these proteins in control treatments.

FIG. 9 shows Western analysis for Raf-1 pathway activation in response to ZM336372 treatment. Total cellular extracts from (A) H727 and (B) BON cells treated with DMSO (Control) and 20 and 100 μmol/L ZM336372 for 2 d. In control H727 and BON cells, there is little activation of the Raf-1/MEK/ERK system by protein phosphorylation; however, with treatment, there are dose-dependent increases in phosphorylated Raf-1 at Ser338 (pRaf-1), MEK 1/2 (pMEK 1/2), and ERK 1/2 (PERK 1/2) indicating Raf-1 pathway activation. C, Western analysis of H727 cells treated at 2, 4, and 6 d with control and 100 Amol/L ZM336372. There is phosphorylation of MEK 1/2 (pMEK 1/2) greater than controls out to 6 d. Samples are loaded equally as shown by G3PDH.

FIG. 10 shows the effect of ZM336372 effect upon chromogranin A (CgA) and hASH1. Total cellular extracts from (A) H727 and (B) BON cells treated with DMSO (Control) and 20 and 100 μmol/L ZM336372 for 2 d. In control H727 and BON cells, there are high levels of both chromogranin A and hASH1. However, with addition of ZM336372, there is a dose-dependent decrease in both markers. C, to determine how quickly the reduction in chromogranin A and hASH1 occur, H727 cells were treated with 100 μmol/L ZM336372 at times of 10 min, 1, 12, 24, and 48 h and harvested and assayed by Western analysis. Reduction in chromogranin A and hASH1 were first recognized at 1-h posttreatment; however, temporal reduction of chromogranin A and hASH1 occurred. D, the lasting effect of chromogranin A reduction was analyzed. Western analysis of H727 treated with 100 Amol/L ZM336372 and control treated (DMSO) at days 2, 4, and 6. There was persistent reduction of chromogranin A to day 6 was seen with treatment. All samples were loaded equally as shown by G3PDH.

FIG. 11 shows the effect of ZM336372 on cell proliferation through a 3,4-(4,5-Dimethylthiazol-2-yl)-2,5-diphenyltetrazolium bromide growth assay. H727 (A) and BON (B) cells were treated with control, DMSO, and 100 μmol/L ZM336372 to days 16 and 10, respectively. Both H727 and BON cell proliferation was inhibited in the presence of drug compared with controls. C, Western analysis of cell cycle inhibitors p21 and p18. H727 cells treated with DMSO or 20 and 100 μmol/L ZM336372 for 2 d. H727 cells without treatment had little or no detectable expression of p21 or p18; however, with treatment, there was significant induction of p21 and p18. G3PDH shows equal loading. D, expression of cell cycle inhibitor p21 in ZM336372-treated carcinoid tumor cells. H727 cells treated with DMSO or 100 μmol/L ZM336372 for 2 d. Pancreatic cancer cells, MiaPaCa2 in that Raf-1 pathway is naturally activated and was used as control without ZM336372 treatment. Cell lysates were obtained and analyzed for Raf-1 pathway activation (pERK1/2) and p21 expression by western blot. H727 cells without treatment had little or no detectable expression of pERK1/2 and p21 proteins whereas treatment by ZM336372 on H727 cells showed high levels of pERK1/2 and p21 proteins. Importantly, MiaPaCa2 cells had no detectable expression of p21 but high levels of pERK1/2 proteins. G3PDH shows equal loading.

FIG. 12 shows cellular toxicity analysis of ZM336372. Cells were treated with increasing concentrations of ZM336372 or equal volume DMSO and normalized to nontreated H727 and BON cells for 2 d in triplicate. Propidium iodide exclusion was analyzed by exclusion on flow cytometry. A, in H727 cells, significant viability was maintained in concentrations used in further analysis in this paper, from 20 to 100 μmol/L ZM336372 compared with DMSO treatment controls. B, furthermore, BON cells maintained f70% viability at high concentrations of ZM336372. Points, averages of three independent experiments; bars, SD.

### DETAILED DESCRIPTION OF PREFERRED EMBODIMENTS

The present invention provides methods of treating NE tumors via the activation of the raf-1 signaling pathway, by inhibiting GSK3 activities. It is surprisingly discovered that inhibition of GSK3 activity or reduction of GSK3 levels lead to inhibition of NE tumor growth or symptoms.

It was previously shown that medullary thyroid cancer cells respond to a ras signal with a differentiation response (Nakagawa et al., 1987, Proc. Natl. Acad. Sci. USA 84:5923-5927). In an in vitro model of medullary thyroid cancer (Chen et al., 1996, Surgery 120:168-172), activation of raf-1 in medullary thyroid cancer cells causes cessation of growth, phenotypic differentiation, and down-regulation of the RET proto-oncogene (Carson-Walter et al., 1998, Oncogene 17:367-376). Similarly, in pheochromocytoma cell lines, induction of ras also results in cessation of cell growth (Wood et al., 1993, Proc. Natl. Acad. Sci. USA 90:5016-5020). In addition, ras/raf-1 activation in small cell lung cancer cells results in suppression of growth capacity, loss of soft agar cloning ability, and cell cycle arrest (Ravi et al., 1999, Am. J. Respir. Cell. Mol. Biol. 20:543-549). These in vitro data suggest that activation of the ras/raf-1 signal transduction pathway can modulate the growth and phenotype of medullary thyroid cancer and small cell lung cancer cells, two prototypic neuroendocrine tumors. However, the role of raf-1 in other NE tumors, especially the more common ones arising in the GI tract, is unknown.

The present investigators have shown NE tumor cells such as pancreatic carcinoid BON, medullary thyroid cancer TT, and pulmonary carcinoid H727 have high basal levels of active, non-phosphorylated GSK3β with very low levels of the inactivated, phosphorylated GSK3β. The present inventors discovered that activation of raf-1 by estradiol-treament of BON-raf, TT-raf, and H727-raf cells led to significant inhibition of GSK3β. This indicates that induction of raf-1 in NE cells resulted in GSK3β inhibition. Based on these discoveries, the present invention provides methods and compositions for reducing NE tumor growth and hormone suppression by inhibiting GSK3β.

In one embodiment, the present invention provides a pharmaceutical composition comprising a GSK3 inhibitor or antagonist, and a pharmaceutically acceptable excipient, for the prevention, inhibition, or treatment of NE tumors. The present invention also provides a method of NE tumor treatment using the pharmaceutical composition.

TABLE 1
Examples of Known GSK3 Inhibitors

| Compound | References |
|---|---|
| Li+ | Klein & Melton, Proc. Natl. Acad. Sci. USA 93, 8455-8459 (1996); |
| | Stambolic et al., Curr. Biol. 6, 1664-1668 (1996); Davies et al., Biochem. |
| | J. 351, 95-105 (2000) |
| SB216763 | 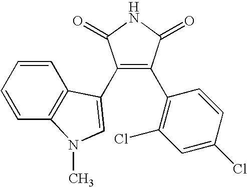 |
| SB415286 | 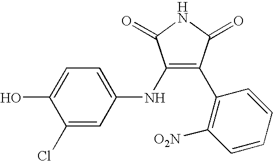 |
| indirubins | LeClerc et al., J. Biol. Chem. 276, 251-260 (2001); Meijer et al., Chem. |
| | Biol. 10, 1255-1266 (2003); |
| | Polychronopoulos et al., J. Med. Chem. 47, 935-946 (2004). |
| Paullones | Leost et al., Eur. J. Biochem. 267, 5983-5994 (2000). |
| Hymenialdisine | Meijer et al., Chem. Biol. 7, 51-63 (1999). |
| Azakenpaullone | Hughes, K., Mikolakaki, E., Plyte, S. E., Totty, N. F. & Woodgett, |
| | J. R. Modulation of the glycogen synthase kinase-3 family by tyrosine |
| | phosphorylation. EMBO J. 12, 803-808 (1993) |
| Thienyl and phenyl α- | Cole et al., Biochem. J. 377, 249-255 (2004). |
| halomethyl ketones | Cohen, Phil. Trans. R. Soc. Lond. B 354, 485-495 (1999). |
| CHR 99021 | Cohen et al., Nature Rev. Mol. Cell. Biol. 2, 769-776 (2001). |
| AR-A014418 | Frame et al., Mol. Cell 7, 1321-1327. |
| Bis-7- | Dajani et al. Cell 105, 721-732 (2001). |
| azaindolylmaleimides | |
| CHR 98023 | Kim, L. & Kimmel, A. R. GSK3, a master switch regulating cell fate |
| | specification and tumorigenesis. Curr. Opin. Genet. Dev. 10, 50-514 |
| | (2000). |
| CHR-98014 | Seidensticker et al., Biochim. Biophys. Acta 1495, 168-182 (2000). |
| ZM336372 | 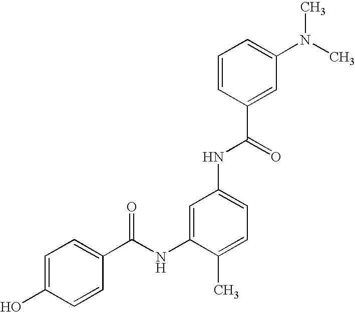 |

Any GSK inhibitor can be used for the preparation of the composition or in the method of the invention. Many such inhibitors are known and readily available to those ordinarily skilled in the art, some are specifically described hereinbelow. See for example Cohen and Coedert, Nature Rev., 2004 3:479-487, which reviews the properties and therapeutic potential of several known GSK3 inhibitors. Some examples of the preferred GSK3 inhibitors are shown in Table 1.

In a preferred embodiment, the present invention provides a pharmaceutical composition comprising a GSK3 inhibitor, or a pharmaceutically acceptable salt thereof, and a pharmaceutically acceptable carrier, at a therapeutically effective concentration, to prevent, inhibit or reverse NE tumors. In one aspect of the invention, GSK3 inhibitor treatment is shown to inhibit growth of cultured NE tumor cells, as well as to inhibit tumor size in whole animals.

In a preferred embodiment, the pharmaceutical composition of the present invention comprises at least one of Li+, SB216763, or ZM336372.

Lithium is a fixed monovalent cation and the lightest of the alkali metals. Li⁺ has the highest energy of hydration of the alkali metals and, as such, can substitute for Na⁺ (and to a lesser extent K⁺) for ion transport by biological systems. Lithium is both electroactive and hydrophilic, and trace amounts of Li⁺ are found in human tissues, for example, the typical human blood plasma concentrations of Li⁺ are about 17 μg/L. Li⁺ has been used for over fifty years in humans as psychotropic drugs such as for mod stabilization, but unlike other psychotropic drugs, Li⁺ has no discernible psychotropic effects in normal man, although the therapeutic efficacy of lithium in the treatment of acute mania and the prophylactic management of bipolar (manic/depressive) disorder has been consistently demonstrated. The oral and parenteral administration of lithium salts, such as lithium carbonate and lithium citrate, has also found widespread use in the current treatment of, for example, alcoholism, aggression, schizophrenia, unipolar depression, skin disorders, immunological disorders, asthma, multiple sclerosis, rheumatoid arthritis, Crohn's disease, ulcerative colitis, and irritable bowel syndrome, as well as for use in many other diseases and conditions. Lithium's main mechanism of action is by altering cation transport across cell membranes in nerve and muscle cells and influencing reuptake of serotonin and/or norepinephrine. It also acts by inhibiting second messenger systems involving the phosphatidylinositol cycle and inhibiting postsynaptic D2 receptor supersensitivity.

When administered to humans, lithium is known to have side effects, such as drowsiness, weakness, nausea, fatigue, hand tremor, or increased thirst and urination. Lithium has a relatively small therapeutic window, and must be used carefully to achieve efficacy while avoiding side effects. This is especially the case when prescribed with medications that can alter lithium concentrations such as diuretics, ACE inhibitors, NSAIDs, neuroleptics, tetracycline, and COX2 inhibitors, and when prescribed for patients with thyroid, kidney, or heart disorders, epilepsy, or brain damage. Those skilled in the art understood that with regular monitoring lithium is a safe and effective drug.

Administration of an “effective amount” or a “therapeutically effective amount” of a GSK-3β inhibitor of the present invention means an amount that is useful, at dosages and for periods of time necessary to achieve the desired result. The therapeutically effective amount of a GSK-3 inhibitor in accordance with the present invention may vary according to factors, such as the disease state, age, sex, and weight of the subject. Dosage regimens of a GSK-3 inhibitor, such as lithium, in the patient may be adjusted to provide the optimum therapeutic response. For example, several divided doses may be administered daily or the dose may be proportionally reduced as indicated by the exigencies of the therapeutic situation. In the context of the present invention, a “pharmaceutically acceptable salt,” refer to salts prepared from pharmaceutically acceptable, non-toxic acids.

The pharmaceutical compositions according to the invention can be present and administered as liquid, semi-solid or solid medicament forms and in the form of e.g. injection solutions, drops, juices, syrups, suspensions, sprays, granules, tablets, pellets, patches, capsules, plasters, suppositories, ointments, creams, lotions, gels, emulsions or aerosols, and comprise, for example, lithium or another GSK-3β inhibitor, pharmaceutical auxiliary substances according to the galenical form, such as e.g. carrier materials, fillers, solvents, diluents, surface-active substances, dyestuffs, preservatives, disintegrating agents, anti-friction agents, lubricants, flavorings and/or binders. These auxiliary substances can be, for example: water, ethanol, 2-propanol, glycerol, ethylene glycol, propylene glycol, polyethylene glycol, polypropylene glycol, glucose, fructose, lactose, sucrose, dextrose, molasses, starch, modified starch, gelatin, sorbitol, inositol, mannitol, microcrystalline cellulose, methylcellulose, carboxymethylcellulose, cellulose acetate, shellac, acetyl alcohol, polyvinylpyrrolidone, paraffins, waxes, naturally occurring and synthetic gums, acacia gum, alginates, dextran, saturated and unsaturated fatty acids, stearic acid, magnesium stearate, zinc stearate, glyceryl stearate, sodium lauryl sulfate, edible oils, sesame oil, coconut oil, ground nut oil, soy bean oil, lecithin, sodium lactate, polyoxyethylene and -propylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crosspovidone, agar and bentonite. The choice of auxiliary materials and the amounts thereof to be employed depend on whether the medicament is to be administered orally, perorally, subcutaneously, parenterally, intravenously, intraperitoneally, intradermally, intramuscularly, intranasally, buccally, rectally or locally, for example to infections on the skin, the mucous membranes and the eyes. Formulations in the form of tablets, coated tablets, capsules, granules, drops, juices and syrups are suitable, inter alia, for oral administration, and solutions, suspensions, easily reconstitutable dry formulations and sprays are suitable for parenteral, topical and inhalatory administration. Lithium or another GSk-3β inhibitor according to the invention in a depot in dissolved form or in a patch, optionally with the addition of agents which promote penetration through the skin, are suitable formulations for percutaneous administration. Formulation forms which can be used orally or percutaneously can release the lithium or another GSk-3β inhibitor according to the invention in a delayed manner.

The medicaments and pharmaceutical compositions according to the invention are prepared with the aid of agents, devices, methods and processes which are well-known in the prior art of pharmaceutical formulation, as described, for example, in “Remington's Pharmaceutical Sciences”, ed. A. R. Gennaro, 17th ed., Mack Publishing Company, Easton, Pa. (1985), in particular in part 8, sections 76 to 93.

Thus for a solid formulation, such as a tablet, the active compound of the medicament, i.e. lithium or another GSk-3β inhibitor, can be mixed with a pharmaceutical carrier, e.g. conventional tablet constituents, such as maize starch, lactose, sucrose, sorbitol, talc, magnesium stearate, dicalcium phosphate or gum, and pharmaceutical diluents, such as e.g. water, in order to form a solid preformulation composition which comprises a compound according to the invention or a pharmaceutically acceptable salt thereof in homogeneous distribution. Homogeneous distribution here is understood as meaning that the active compound is distributed uniformly over the entire preformulation composition, so that this can easily be divided into unit dose forms of the same action, such as tablets, pills or capsules. The solid preformulation composition is then divided into unit dose forms. The tablets or pills of the medicament according to the invention or of the compositions according to the invention can also be coated, or compounded in another manner in order to provide a dose form with delayed release. Suitable coating compositions are, inter alia, polymeric acids and mixtures of polymeric acids with materials such as e.g. shellac, acetyl alcohol and/or cellulose acetate.

The amount of active compound to be administered to the patient varies and depends on the weight, age and disease history of the patient, as well as on the mode of administration, the indication and the severity of the disease. 0.1 to 5,000 mg/kg, in particular 1 to 500 mg/kg, preferably 2 to 250 mg/kg of body weight of lithium or another GSK-3β inhibitor according to the invention are usually administered.

Antibodies

In another embodiment, this invention provides neutralizing antibodies to inhibit the biological action of GSK3 protein. An antibody suitable for the present invention may be a polyclonal antibody. Preferably, the antibody is a monoclonal antibody. The antibody may also be isoform-specific. The monoclonal antibody or binding fragment thereof of the invention may be Fab fragments, F(ab)2 fragments, Fab′ fragments, F(ab′)2 fragments, Fd fragments, Fd′ fragments or Fv fragments. Domain antibodies (dAbs) (for review, see Holt et al., 2003, Trends in Biotechnology 21:484-490) are also suitable for the methods of the present invention.

Anti-GSK3 antibodies are known to those skilled in the art and some are available commercially (e.g. from Santa Cruz Biotechnology Inc., Santa Cruz, Calif.; Chemicon International, Inc., Temecula, Calif., and Cell Signaling Technology, Inc., Beverly, Mass.). In addition, various methods of producing antibodies with a known antigen are well-known to those ordinarily skilled in the art (see for example, Harlow and Lane, 1988, Antibodies: A Laboratory Manual. Cold Spring Harbor Laboratory Press, Cold Spring Harbor, N.Y.; see also WO 01/25437). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In accordance with the present invention, the antibodies or binding fragments thereof include those which are capable of specific binding to a target protein or an antigenic fragment thereof, preferably an epitope that is recognized by an antibody when the antibody is administered in vivo. Antibodies can be elicited in an animal host by immunization with a target protein-derived immunogenic component, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

The antibodies may be from humans, or from animals other than humans, preferably mammals, such as rat, mouse, guinea pig, rabbit, goat, sheep, and pig, or avian species such as chicken. Preferred are mouse monoclonal antibodies and antigen-binding fragments or portions thereof. In addition, chimeric antibodies and hybrid antibodies are embraced by the present invention. Techniques for the production of chimeric antibodies are described in e.g. Morrison et al., 1984, Proc. Natl. Acad. Sci. USA, 81:6851-6855; Neuberger et al., 1984, Nature, 312:604-608; and Takeda et al., 1985, Nature, 314:452-454. For human therapeutic purposes, humanized, or more preferably, human antibodies are used.

Further, single chain antibodies are also suitable for the present invention (e.g., U.S. Pat. Nos. 5,476,786 and 5,132,405 to Huston; Huston et al., 1988, Proc. Natl. Acad. Sci. USA, 85:5879-5883; U.S. Pat. No. 4,946,778 to Ladner et al.; Bird, 1988, Science, 242:423-426 and Ward et al., 1989, Nature, 334:544-546). Single chain antibodies are formed by linking the heavy and light immunoglobulin chain fragments of the Fv region via an amino acid bridge, resulting in a single chain polypeptide. Univalent antibodies are also embraced by the present invention.

Many routes of delivery are known to the skilled artisan for delivery of antibodies. For example, direct injection may be suitable for delivering the antibody to the site of interest. It is also possible to utilize liposomes with antibodies in their membranes to specifically deliver the liposome to the area where target gene expression or function is to be inhibited. These liposomes can be produced such that they contain, in addition to monoclonal antibody, other therapeutic agents, such as those described above, which would then be released at the target site (e.g., Wolff et al., 1984, Biochem. et Biophys. Acta, 802:259).

Antisense Nucleic Acid Molecules

In another embodiment of the invention, GSK3 inhibitors suitable for the present invention are antisense oligonucleotides. The expression, preferably constitutively, of antisense RNA in cells has been known to inhibit gene expression, possibly via blockage of translation or prevention of splicing. In this regard, interference with splicing allows the use of intron sequences which should be less conserved and therefore result in greater specificity, inhibiting expression of a gene product of one species but not its homologue in another species.

The term antisense component corresponds to an RNA sequence as well as a DNA sequence, which is sufficiently complementary to a particular mRNA molecule, for which the antisense RNA is specific, to cause molecular hybridization between the antisense RNA and the mRNA such that translation of the mRNA is inhibited. Such hybridization can occur under in vivo conditions. This antisense molecule must have sufficient complementarity, about 18-30 nucleotides in length, to the target gene so that the antisense RNA can hybridize to the target gene (or mRNA) and inhibit target gene expression regardless of whether the action is at the level of splicing, transcription, or translation. The antisense components of the present invention may be hybridizable to any of several portions of the target cDNA, including the coding sequence, 3′ or 5′ untranslated regions, or other intronic sequences, or to target mRNA. The coding region for GSK3B is well-known and well characterized (see e.g. Woodgett, 1990, EMBO Journal 9(8):2431-2438; and Goode et al., 1992). J. Biol. Chem. 267 (24): 16878-16882. One of ordinary skills in the art will readily recognize that the antisense molecules can be easily designed based on the known mRNA sequences.

Antisense RNA is delivered to a cell by transformation or transfection via a vector, including retroviral vectors and plasmids, into which has been placed DNA encoding the antisense RNA with the appropriate regulatory sequences including a promoter to result in expression of the antisense RNA in a host cell. In one embodiment, stable transfection and constitutive expression of vectors containing target cDNA fragments in the antisense orientation are achieved, or such expression may be under the control of tissue or development-specific promoters. Delivery can also be achieved by liposomes.

For in vivo therapy, the currently preferred method is direct delivery of antisense oligonucleotides, instead of stable transfection of an antisense cDNA fragment constructed into an expression vector. Antisense oligonucleotides having a size of 15-30 bases in length and with sequences hybridizable to any of several portions of the target cDNA, including the coding sequence, 3′ or 5′ untranslated regions, or other intronic sequences, or to target mRNA, are preferred. Sequences for the antisense oligonucleotides to target are preferably selected as being the ones that have the most potent antisense effects. Factors that govern a target site for the antisense oligonucleotide sequence include the length of the oligonucleotide, binding affinity, and accessibility of the target sequence. Sequences may be screened in vitro for potency of their antisense activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides.

The preferred target antisense oligonucleotides are those oligonucleotides which are stable, have a high resistance to nucleases, possess suitable pharmacokinetics to allow them to traffic to target tissue site at non-toxic doses, and have the ability to cross through plasma membranes.

Phosphorothioate antisense oligonucleotides may be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNase H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

The delivery route will be the one that provides the best antisense effect as measured according to the criteria described above. In vitro and in vivo assays using antisense oligonucleotides have shown that delivery mediated by cationic liposomes, by retroviral vectors and direct delivery are efficient. Another possible delivery mode is targeting using antibody to cell surface markers for the target cells. Antibody to target or to its receptor may serve this purpose.

RNAi

In a further embodiment, the antagonizing agent is small interfering RNAs (siRNA, also known as RNAi, RNA interference nucleic acids). siRNA are double-stranded RNA molecules, typically 21 n.t. in length, that are homologous to the target gene and interfere with the target gene's activity.

siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide siRNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48; Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411:428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerheadribozyme compositions of the invention (see below).

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:863-74). The effectiveness of the siRNA may be assessed by any of a number of assays following introduction of the dsRNAs. These include Western blot analysis using antibodies which recognize the target gene product following sufficient time for turnover of the endogenous pool after new protein synthesis is repressed, reverse transcriptase polymerase chain reaction and Northern blot analysis to determine the level of existing target mRNA.

Further compositions, methods and applications of siRNA technology are provided in U.S. patent application Nos. 6,278,039, 5,723,750 and 5,244,805, which are incorporated herein by reference.

Other Inhibition Approaches

Alternatively, other nucleic acid sequences which inhibit or interfere with gene expression (e.g., ribozymes, triplex nucleic acids, DNA enzymes, aptamers) can be used to inhibit or interfere with the activity of RNA or DNA encoding a GSK3 protein.

Ribozymes are enzymatic RNA molecules capable of catalyzing specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or siRNA are substantially the same sequence of at least 5 and preferably 6, 7, 8, 9, 10, 11, 12, 13, 14, 15, 16, 17, 18, 19 or 20 or more contiguous nucleotides of a target nucleic acid.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C., 1991, Anticancer Drug Des., 6:569-84; Helene, C., et al., 1992, Ann. N.Y. Acad. Sci., 660:27-36; and Maher, L. J., 1992, Bioassays 14:807-15). Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of target gene. DNA enzymes incorporate some of the mechanistic features of both antisense and ribozyme technologies. DNA enzymes are designed so that they recognize a particular target nucleic acid sequence, much like an antisense oligonucleotide. They are, however, catalytic and specifically cleave the target nucleic acid. There are currently two basic types of DNA enzymes, both of which were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

The following examples are intended to illustrate preferred embodiments of the invention and should not be interpreted to limit the scope of the invention as defined in the claims.

### EXAMPLES

### Example 1

### LiCl Increases Phosphorylation of GSK-3β

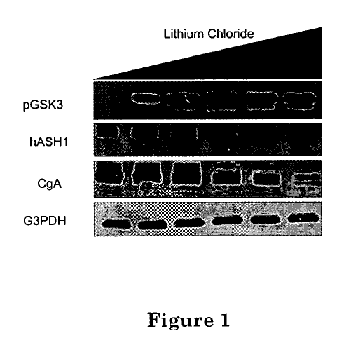

TT cells were treated with varying concentrations of LiCl (0-50 μM). Treatment of NE tumor cells with lithium chloride resulted in inhibition of GSK-3β as shown by increasing levels of phosphorylated GSK-3β (FIG. 1).

### Example 2

### Lithium Chloride Suppresses Tumor Cell Growth In Vitro

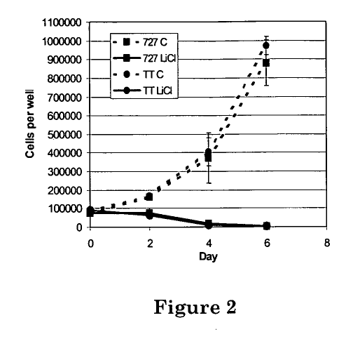

FIG. 2 Treatment of medullary thyroid cancer TT and carcinoid H727 cells with lithium chloride (LiCl 20 mM, solid lines) inhibited cellular growth compared to control (C) cells (dotted lines).

### Example 3

### Lithium Chloride Suppresses Tumor Cell Growth In Vivo

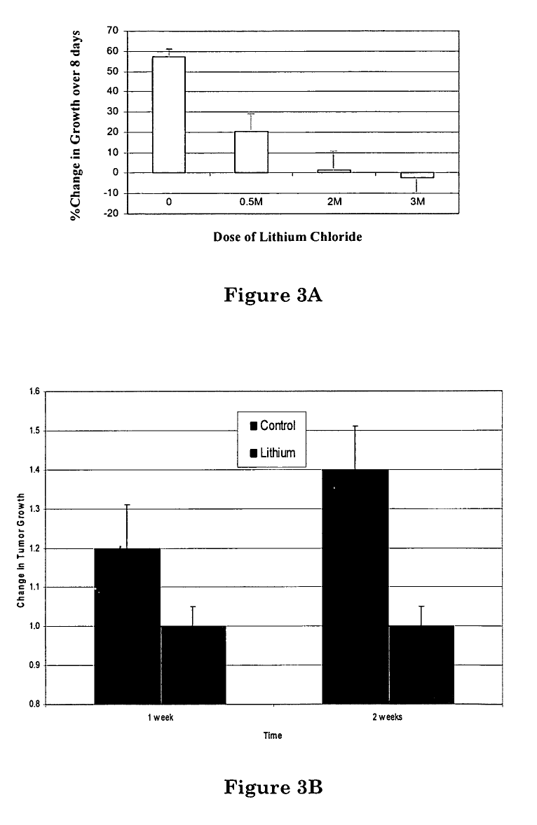

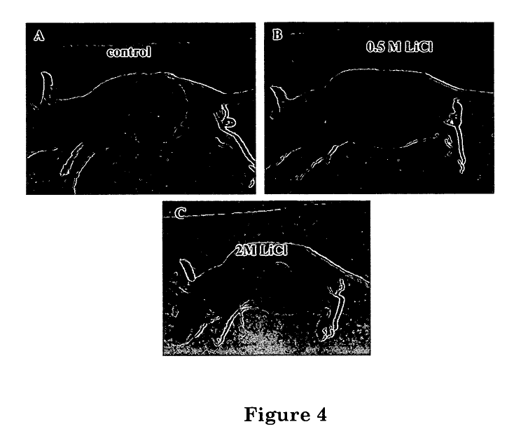

Sixty male nude mice (Strain BABL/C) were injection with 10⁷ human carcinoid H727 cells in the flank. After 3 weeks, measurable NE tumors were found in the mice. The animals were then treated with varying dosages (100 μl of 0, 0.5, 2 and 3 M) lithium chloride by intraperitoneal injection every 2 days for a total of 10 treatments. The lithium treatment lead to a significant reduction in NE tumor growth (FIGS. 3A and 4). FIG. 3B further shows the results of 400 μg treatment for 2 weeks. Importantly, the marked reduction in tumor growth and hormone production was not associated with any measurable toxicity in the animals.

Similar results were obtained with nude mice injected with medullary thyroid cancer cells.

### Example 4

### Lithium Chloride Reduces NE Hormone Production

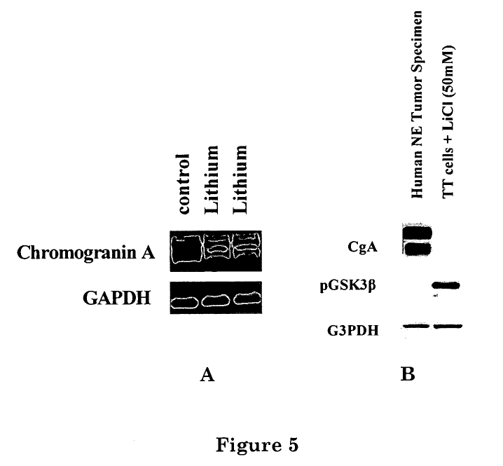

TT cells were treated with varying concentrations of LiCl (0-50 μM). Lithium chloride treatment led to a reduction to NE hormone production (FIG. 1). The lithium treatment lead to suppression the NE marker chromogranin A (FIG. 5A).

FIG. 5B further shows a similar experiment using a human GI NE tumor sample. which showed high levels of CgA but no pGSK3B. As hypothesized, the human GI NE tumor lacked phosphorylated GSK3B compared to control human MTC TT cells treated with lithium chloride. Furthermore, human NE tumors are known to lack phosphorylated ERK1/2 compared to normal tissues and non-NE tumors by Western blot. As demonstrated by FIG. 5B, treatment of NE TT cells with Lithium leads to high levels of pGSK3B and suppression of CgA.

### Example 5

### Clinical Tests of Lithium Salts for Treating NE tumors

Three-month progression-free survival of patients with low-grade neuroendocrine tumors treated with lithium carbonate are tested clinically. The response rate and overall survival of patients, the effect of lithium carbonate on NE-tumor specific markers (e.g. Chromogranin A, 5-HIAA, gastrin, etc.), and the toxicity and tolerability of lithium carbonate in patient population are determined.

1. Eligibility Criteria:

a. Histologically confirmed metastatic low-grade neuroendocrine neoplasms. Small cell lung cancers, paragangliomas and pheochromocytomas are excluded. Pathologic diagnosis is confirmed.

b. Measurable disease and radiographic evidence of disease progression following any prior systemic therapy, chemoembolization, bland embolization, or observation. Disease progression is defined as either of the following documented by comparison of the on-study radiographic assessment with a prior assessment of the same type within the precious 52 calendar weeks: Appearance of a new lesion or at least 20% increase in the longest diameter (LD) of any previously documented lesion or an increase in the sum of the LDs of multiple lesions in the aggregate of 20%.

c. Four weeks from the completion of major surgery, chemotherapy, or other systemic therapy or local liver therapy to study registration.

d. The following laboratory values obtained within 7 days prior to registration: Absolute neutrophils count (ANC) ≧1000/mm³, Platelets ≧75,000/mm3, Hemoglobin ≧8.0 g/dL, Total bilirubin ≦2.0× the upper limit of normal (ULN), AST ≦3×ULN or ≦5×ULN if liver metastases are present, Creatinine≦ULN, Serum sodium within normal limits.

e. ECOG performance status of ≦2.

f. Capable of understanding the investigational nature, potential risks and benefits of the study and able to provide valid informed consent.

g. Availability of tissue specimens to be analyzed for Ki-67 and pathologic confirmation. A portion of the tumor block is preferred.

h. Age ≧18 years. Children are excluded from this study but will be eligible for future pediatric trials, if applicable.

i. Women must not be pregnant or lactating due to the deleterious effects of lithium carbonate on a fetus or small child. All females of childbearing potential must have a blood test or urine study within 2 weeks prior to registration to rule out pregnancy.

j. Patients are not allowed to be on concurrent chemotherapy or radiation therapy.

k. Patients are excluded if they have any of the following: Gastrointestinal tract disease resulting in an inability to take oral medication (i.e. ulcerative disease, uncontrolled nausea, vomiting, diarrhea, bowel obstruction, or inability to swallow the tablets; history of hypothyroid disease; Significant, active cardiac disease; Patients must not be taking the following medications: diuretics, ACE inhibitors, NSAIDs (except aspirin or sulindac), neuroleptics, tetracycline, and COX2 inhibitors

2. Treatment Plan

Lithium carbonate is dosed on a flat scale of mg/day and not by weight or body surface area (BSA). Lithium carbonate is provided as a 300 mg tablet and supplied in bottles of 90 tablets each. The starting dose of lithium carbonate is 300 mg orally three times daily.

Lithium carbonate is started within 7 days of registration and is taken daily without breaks in treatment. Each cycle will be defined as 28 days.

Tablets are taken with meals to reduce GI upset. A serum lithium level is checked after 4-5 days of treatment by drawing a blood sample prior to the morning dose of lithium. The target lithium level is 0.8 and 1.0 mmol/L. If the lithium level is less than 0.8 mmol/L, the lithium level is increased by 300 mg/day, added as an extra tablet in the morning, afternoon, or evening dose. The lithium level is again determined 4-5 days later prior to the morning dose of lithium on the fifth day after the adjustment. This process continues until the target lithium level has been achieved. At that point, lithium levels are monitored weekly for four weeks and then monthly while on a stable dose of lithium.

3. Monitoring Toxicity and Re-Treatment

All scheduled subsequent doses of lithium withheld from patients who experience any of the following unacceptable toxicities thought to be treatment related during a treatment course: CTC grade 4 hypertension, CTC grade 4 ataxia or dizziness, CTC grade 4 neutropenia or platelet count, AST or ALT >5×ULN, CTC grade 3 bilirubin elevations, CTC grade 3 ataxia or dizziness lasting >10 days with continued dosing of LITHIUM, Any CTC grade 3 toxicity that is thought to be related to study drug (including nausea/vomiting not controlled by anti-emetics), CTC grade 2 hypo or hypernatremia, CTC grade 2 hematuria, or Serum creatinine 1.5×ULN.

Lithium is also withheld at doctor's discretion if the patient is experiencing any other CTC grade 3 toxicity and it is not feasible to continue the study. If the administration is interrupted due to unacceptable toxicities, the patient is evaluated at least once a week following demonstration of the toxicity until either resolution of the toxicity occurs, the patient comes off study, or until retreatment begins. Retreatment begins once the toxicity has resolved to Grade 1 or less prior to the administration of subsequent doses. If toxicities have failed to resolve to Grade 1 or less within 28 days, the participant is removed from the study.

If any of the toxicities described above occur and are thought to be treatment related, then the dose of lithium is withheld. Subsequent treatment with lithium only begins once the toxicities have resolved to a CTC grade 1 toxicity or less (or baseline value for AST/ALT). The subsequent treatment resumes at dose level −1. If a second episode of this or any other toxicity occurs and is thought to be treatment related, then the patient may receive another dose reduction to dose level −2. If an unacceptable toxicity occurs at dose level −2, then the lithium must be permanently discontinued. If a patient requires a dose delay of >3 weeks, then the treatment will be permanently discontinued.

TABLE 2
Dose reduction steps for Lithium

| | Dose Reduction ¹ | | |
|---|---|---|---|
| | Dose Level -1 | Dose Level -2 | Dose Level -3 |
| Lithium | 300 mg/day | 300 mg/day | Discontinue lithium |
| | decrease of dose | decrease of dose | |
| | at which there was | at which there was | |
| | toxicity | toxicity | |

Dose reduction should be based on the worst toxicity demonstrated at the last dose.
Dose reduction below level -3 mg is not allowed

4. Duration of Treatment and Follow-Up

Patients receive protocol therapy unless: 1) Extraordinary Medical Circumstances: If at any time the constraints of this protocol are detrimental to the patient's health, protocol treatment is discontinued; 2) Patient withdraws consent; 3) The patient has progression of disease as defined below; 4) The patient has a toxicity as described in section 1.b. that requires a discontinuation from further treatment and the toxicity does not resolve in >28 days; 5) Any severe toxicity thought secondary to the study medication that, in the investigator's opinion, retreatment would endanger the health of the patient.

For this protocol, all patients, including those who discontinue protocol therapy early, are followed for response until progression and for survival for 10 years from the date of registration. All patients are followed through completion of all protocol therapy.

5. Measurement of Effect

Solid Tumor Response Criteria (RECIST). To assess objective response, it is necessary to estimate the overall tumor burden at baseline to which subsequent measurements will be compared. Measurable disease is defined by the presence of at least one measurable lesion. All measurements should be recorded in metric notation by use of a ruler or calipers. The same method of assessment and the same technique should be used to characterize each identified lesion at baseline and during follow-up. All baseline evaluations should be performed as closely as possible to the beginning of treatment and never more than four weeks before registration. At baseline, tumor lesions will be characterized as either measurable or non-measurable.

I) Measurable. Lesions that can be accurately measured in at least one dimension (longest diameter to be recorded) as >20 mm (2.0 cm) with conventional techniques or as >10 mm (1.0 cm) with spiral CT scan. If the measurable disease is restricted to a solitary lesion, its neoplastic nature should be confirmed by cytology/histology.

II) Non-Measurable All other lesions, including small lesions [longest diameter <20 mm (2.0 cm) with conventional techniques or <10 mm (1.0 cm) with spiral CT scan] and truly non-measurable lesions. Lesions considered to be truly non-measurable include the following: bone lesions, leptomeningeal disease, ascites, pleural/pericardial effusion, lymphangitis cutis/pulmonis, abdominal masses that are not confirmed and followed by imaging techniques, and cystic lesions.

2) Definitions of Response: Measurable disease. All measurable lesions up to a maximum of five lesions per organ and 10 lesions in total, representative of all involved organs. Target lesions should be selected on the basis of their size (those with the longest diameters) and their suitability for accurate repeated measurements. The sum of the longest diameters of all target lesions will be calculated at baseline and reported as the baseline sum longest diameter. The sum longest diameter will be used to characterize the objective tumor response. For lesions measurable in 2 or 3 dimensions, always report the longest diameter at the time of each assessment.

I. Complete Response (CR). The disappearance of all target lesions. To be assigned a status of complete response, changes in tumor measurements must be confirmed by repeat assessments performed no less than four weeks after the criteria for response are first met.

II. Partial Response (PR). At least a 30% decrease in the sum of the longest diameters of target lesions, taking as reference the baseline sum longest diameter. To be assigned a status of partial response, changes in tumor measurements must be confirmed by repeat assessments performed no less than four weeks after the criteria for response are first met.

III. Progressive Disease (PD). At least a 20% increase in the sum of the longest diameters of target lesions, taking as reference the smallest sum longest diameter recorded since the baseline measurements, or the appearance of one or more new lesion(s).

IV. Stable Disease (SD). Neither sufficient shrinkage to qualify for partial response nor sufficient increase to qualify for progressive disease. To be assigned a status of stable disease, measurements must have met the stable disease criteria at least once after study entry at a minimum interval of 12 weeks.

3) Definitions of Response: Non-measurable disease. All other lesions or sites of disease. Measurements of these lesions are not required, but the presence or absence of each should be noted throughout follow-up.

I. Complete Response (CR). The disappearance of all non-target lesions and normalization of tumor marker levels, if applicable. To be assigned a status of complete response, changes in tumor measurements must be confirmed by repeat assessments performed no less than four weeks after the criteria for response are first met.

II. Incomplete Response/Stable Disease (SD. The persistence of one or more non-target lesion(s) and/or the maintenance of tumor marker levels above the normal limits. To be assigned a status of stable disease, measurements must have met the stable disease criteria at least once after study entry at a minimum interval of eight weeks.

III. Progressive Disease (PD). The appearance of one or more new lesion(s) and/or unequivocal progression of existing non-target lesions.

4) Evaluation of Patient's Best Overall Response. The best overall response is the best response recorded from registration until disease progression/recurrence, taking as reference for progressive disease the smallest measurements recorded since registration. The table below provides overall responses for all possible combinations of tumor responses in target and non-target lesions, with or without new lesions. To be assigned a status of complete or partial response, changes in tumor measurements must be confirmed by repeat assessments performed no less than four weeks after the criteria for response are first met. To be assigned a status of stable disease, measurements must have met the stable disease criteria at least once after study entry at a minimum interval of eight weeks.

TABLE 3
Overall Response for all Possible
Combinations of Tumor Response

| Target Lesions | Nontarget Lesions | New Lesions | Overall Response |
|---|---|---|---|
| CR | CR | No | CR |
| CR | Incomplete | No | PR |
| | response/SD | | |
| PR | Non-PD | No | PR |
| SD | Non-PD | No | SD |
| PD | Any | Yes or No | PD |
| Any | PD | Yes or No | PD |
| Any | Any | Yes | PD |

The First Documentation of Response will be the time between initiation of therapy and first documentation of PR or CR. To be assigned a status of complete or partial response, changes in tumor measurements must be confirmed by repeat assessments performed no less than four weeks after the criteria for response are first met. The duration of overall response will be the period measured from the time that measurement criteria are met for complete or partial response (whichever status is recorded first) until the first date that recurrent or progressive disease is objectively documented, taking as reference the smallest measurements recorded since treatment started. The Duration of Overall Complete Response will be the period measured from the time measurement criteria are met for complete response until the first date that recurrent disease is objectively documented.

5) Survival. Survival will be measured from the date of entry on study. Progression Free Survival will be measured from the date of entry on the study to the appearance of new metastatic lesions or objective tumor progression.

6) Methods of Measurement Imaging based evaluation is preferred to evaluation by clinical examination. The same imaging modality must be used throughout the study to measure disease. CT and magnetic resonance imaging (MRI) are the best currently available and most reproducible methods for measuring target lesions. Conventional CT and MRI should be performed with contiguous cuts of 10 mm or less in slice thickness. Spiral CT should be performed by use of a 5 mm contiguous reconstruction algorithm. This specification applies to tumors of the chest, abdomen, and pelvis, while head and neck tumors, and those of the extremities require specific procedures. Lesions on chest x-ray are acceptable as measurable lesions when they are clearly defined and surrounded by an aerated lung. However, CT is preferable. Tumor markers alone cannot be used to assess response. If initially above the upper normal limit, a tumor marker must return to normal levels for a patient to be considered in complete clinical response when all tumor lesions have disappeared. Clinically detected lesions will only be considered measurable when they are superficial (e.g., skin nodules and palpable lymph nodes). For skin lesions, documentation by color photography, including a ruler to estimate size of the lesion, is recommended. Photographs should be retained at the institution. Cytologic and histologic techniques can be used to differentiate between complete and partial response in rare cases. Cytologic confirmation of the neoplastic nature of any effusion that appears or worsens during treatment is required when the measurable tumor has met response or stable disease criteria. Ultrasound may be used only as an alternative to clinical measurements for superficial palpable lymph nodes, subcutaneous lesions, and thyroid nodules, and for confirming complete disappearance of superficial lesions usually assessed by clinical examination.

7) Biochemical Markers of Response. This evaluation will be performed for descriptive purposes only and will not be used to evaluate the primary endpoint of 3 month progression free survival. Biochemical progression without the demonstration of objective progression of target or nontarget lesions will not be sufficient indication to remove the patient form the study. Patients will have tumor markers specific to their disease drawn prior to enrollment on study and at the beginning of each cycle. We will characterize changes in tumor marker level as: biochemical complete response (bCR), biochemical partial response (bPR), biochemical stable disease (bSD), and biochemical progression of disease (bPD). These are defined as follows: bCR—Normalization of the tumor marker for 2 successive evaluations at least two weeks apart; bPR—Decrease in tumor marker value by ≧50% (without normalization) for two successive evaluations; bSD—Patients who do not meet the criteria for bPR or bCR for at least 90 days on study (Week 12) will be considered stable; bPD—Two consecutive increases in tumor marker to ≧50% above.

6. Study Parameters

Prestudy scans and x-rays used to assess all measurable or non-measurable sites of disease are done within 4 weeks prior to randomization/registration. Prestudy CBC (with differential and platelet count) should be done not more than 4 weeks before randomization/registration. All required prestudy chemistries should be done not more than 4 weeks before randomization/registration—unless specifically required on Day 1 as per protocol.

7. Drug Formulation and Statistics

The primary objective of this study is to determine the proportion of patients who are alive and free from progression of disease in neuroendocrine tumors at 3 months. A drug with minimal activity would be expected to have a rate at 3 months of 15%. Alternatively, Lithium will be considered worthy of further study if its true progression-free survival rate at 3 months is 32% or better. Given that Lithium treatment proposed in this protocol is a novel approach and has been previously untested we would wish to stop the trial early if there is evidence that the therapy is ineffective. We therefore propose a two-stage design.

The initial accrual phase will then consist of entering 30 patients, 28 of whom are expected to be eligible. If fewer than 5 of the initial 28 patients are alive and free from progression of disease at 3 months, the study will cease and the treatment will be abandoned. If 5 or more patients are alive and free from progression of disease at 3 months, 17 additional patients will be accrued of whom 14 are expected to be eligible, for an accrual of 42 eligible patients. If at least 10 among the 42 eligible patients are alive and free from progression at 3 months, the regimen will be considered promising. This two stage design has at least 90% power to detect a 3 month PFS rate of at least 32% against the null of 15% while maintaining a one-sided type I error rate of less than 10%. Table 4 shows the characteristics of the two-stage design where the probability of stopping early is less than 5% if the true 3-month PFS rate is at least 32%.

TABLE 4
Operating Characteristics of the Two-stage Design

| | True Progression-Free Rate at 3 Months | | | | | |
|---|---|---|---|---|---|---|
| | 0.1 | 0.15 | 0.2 | 0.25 | 0.3 | 0.32 |
| Probability of stopping | 0.86 | 0.59 | 0.32 | 0.14 | 0.05 | 0.03 |
| early (<5 PFS at | | | | | | |
| 3 months out of 28) | | | | | | |
| Overall probability of | 0.99 | 0.91 | 0.68 | 0.38 | 0.16 | 0.10 |
| rejecting the treatment | | | | | | |

Assuming that a sufficient number patients will be progression free and alive at 3-months to allow full accrual in the two stage design, Table 5 gives 90% two stage confidence intervals for the true proportion remaining alive and progression free at 3 months. For example, if 16 out of 42 eligible patients are alive and progression-free at 3 months, the 90% confidence interval for the true progression-free survival rate is (26%, 52%). The 90% confidence interval for the true PFS rate will be no wider than 27%.

TABLE 5
90% Confidence Intervals for the True PFS Rate at 3 months

| Observed Number of Patients | Observed | 90% Confidence Interval |
|---|---|---|
| Alive and Progression-free | PFS Rate | for True but Unknown |
| at 3 Months | at 3 Months | PFS Rate at 3 Months |
| 10 | 24% | (14%, 37%) |
| 12 | 29% | (17%, 42%) |
| 14 | 33% | (21%, 47%) |
| 16 | 38% | (26%, 52%) |
| 17 | 41% | (28%, 54%) |
| 18 | 43% | (30%, 57%) |
| 19 | 45% | (32%, 59%) |

Secondary analyses of progression-free survival, overall survival and response rate of Lithium as outlined and validated by the Response Evaluation Criteria in Solid Tumors (RECIST) Group will be exploratory in nature. The response rate of Lithium will be reported and Kaplan-Meier method will be used to estimate both overall and progression-free survival. All patients who receive treatment regardless of eligibility will be evaluated for toxicity and tolerability of Lithium. Table 6 gives the exact 90% confidence limits for the true rate of any grade 3 or higher toxicity assuming that all 47 patients accrued provide toxicity data. The 90% confidence interval for toxicity will be no wider than 26%.

The rate of accrual is about 15 patients/year, and the trial closes in 3.1 years.

TABLE 6
90% Confidence Intervals for Grade 3 or Higher Toxicity

| Observed Number of | | 90% Confidence |
|---|---|---|
| Grade 3 or Worse | Observed | Interval for the |
| Toxicities | Toxicity Rate | Toxicity Rate |
| 2 | 4% | (1%, 13%) |
| 5 | 11% | (4%, 21%) |
| 6 | 13% | (6%, 24%) |
| 8 | 17% | (9%, 29%) |
| 12 | 26% | (15%, 38%) |
| 16 | 34% | (23%, 47%) |
| 20 | 43% | (30%, 56%) |
| 24 | 51% | (39%, 64%) |

### Example 6

### Effect of LiCl on GSK3B Expression in NE Tumors

Several NE cell lines previously characterized and all showed high levels of the active GSK3B protein and low amounts of the inactive phosphorylated GSK3B signaling (Chen et al., Differentiation of medullary thyroid cancer by C-Raf-1 silences expression of the neural transcription factor human achaete-scute homolog-1. Surgery 1996, 120:168-172; and Chen et al., Human achaete-scute homolog-1 is highly expressed in a subset of neuroendocrine tumors. Oncology Reports 1997; 4:775-778). However, the degree of GSK3B signaling in NE tumor samples remains unknown. The NE tumor samples from patients enrolled in the above clinical study are analyzed for expression GSK3B signaling pathway members before and during lithium therapy.

a. Biological Sample Submissions

An informed consent MUST be signed prior to the submission of any biological material. Tissue samples for optional correlative studies should be submitted only from patients who have given written consent for the use of their samples for these purposes. Samples must be submitted for analysis to determine patient eligibility as outlined in section D.1.a. Samples must be submitted prior to registration to confirm eligibility.

As shown in Section D.1.e., patient will undergo tumor biopsies prior to lithium treatment, and periodically after achieving therapeutic serum lithium levels. The NE tumor tissue will be analyzed as detailed below.

b. Expression of GSK3B Pathway Components.

To determine the levels of GSK3B pathway components in NE tumors, immuno-histochemistry with antibodies against GSK3B, phospho-GSK3B, and B-Catenin is used. The levels of the NE markers chromogranin A, HASH1, and serotonin/calcitonin in these tumors are determined

Although protein analysis is preferred due to the ability to determine levels of phosphorylated protein, mRNA levels may also be determined as an alternative for each of the factors with either real time PCR or Northern analysis. Human NE tumor samples have high levels of GSK3B present in NE tumors but low amounts of phosphorylated GSK3B prior to lithium treatment. Furthermore, less aggressive tumors have slight inhibition of GSK3B. Characterizing the expression patterns of these factors may allow more accurate determination of prognosis, and the data from these analyses may predict which patients may be more likely to respond to lithium and other GSK inhibiting compounds.

### Example 7

### Treatment of Human Medullary Thyroid Cancer Cells with SB216763 Led to Reduction of CgA and hASH1

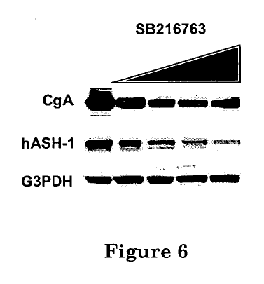

Treatment of MTC TT cells with increasing doses (0-50 μM) of SB216763, a well-characterized GSK3B inhibitor, also resulted in suppression of TT cell growth and in the NE markers chromogranin A production and hASH1 (FIG. 6).

### Example 8

### Treatment of BON and TT Cells with ZM33762 Resulted in High Levels of Phosphorylated GSK3B

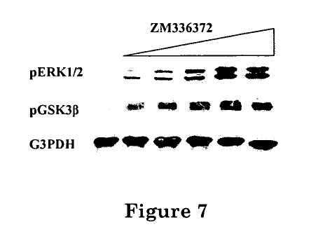

As shown in FIG. 7, treatment of NE tumor cells with ZM336372 (0-50 μM) led to increased levels of phosphorylated GSK3B and ERK1/2.

### Example 9

### A Mouse Model of NE Tumor Progression and Liver Metastasis

The present inventors have developed a mouse model of NE tumor progression and liver metastasis, utilizing nude mice and intrasplenic injection of human NE tumor cells. BON, H727 or TT cells (107) were injected into the nude mouse spleens. The animals underwent distal hemisplenectomies 1 minute later, resecting the injection site. Visible tumors are present in the liver within 8 weeks. By 12 weeks after injection, the tumors occupy almost 50% of the liver, with multiple metastatic lesions. After development of carcinoid liver metastases, serotonin levels in the serum of these mice are markedly elevated. Therefore, these mice are an animal model of the carcinoid syndrome. To determine if carcinoid liver metastases and/or the high levels of circulating serotonin lead to any functional consequences in these animals, the hearts of the mice were serially sectioned before tumor cell injection and 14 weeks after injection, when frank liver metastases were present. While the right ventricle and tricuspid valve from untreated mice appear histologically normal, the hearts from mice with carcinoid liver metastases have a significant amount of fibrosis and scarring with in the ventricle and valves.

### Example 10

### ZM336372, a GSK3 Inhibitor, Suppresses Growth and Neuroendocrine Hormone Levels in Carcinoid Tumor Cells

Materials and Methods

Cell Culture

Human pulmonary carcinoid cells (NCI-H727) were obtained from American Type Culture Collection (Manassas, Va.) and human pancreatic carcinoid tumor cells, BON, a generous gift of Drs. Evers and Townsend (Department of Surgery, University of Texas, Galveston, Tex.; ref. 15), were maintained in RPMI 1640 and DMEM/F12 (Life Technologies, Rockville, Md.), respectively, supplemented with 10% fetal bovine serum (Sigma, St. Louis, Mo.), 100 IU/mL penicillin and 100 Ag/mL streptomycin (Life Technologies) in a humidified atmosphere of 5% CO₂ in air at 37° C.

BON-raf Cells Maintenance and Raf-1 Activation

BON-raf cells were created and maintained and Raf-1 was activated essentially as described (Sippel & Chen, Surgery 2002;132:1035-9; Sippel et al., Am J Physiol Gastrointest Liver Physiol 2003;285:G245-54; McMahon, Methods Enzymol 2001;332:401-17). Briefly, BON cells were stably transduced with the retroviral vector pLNCΔraf:ER to create BON-raf cells. This construct contains estrogen receptor fused with c-raf kinase domain. BON-raf cells were maintained in DMEM/F12 supplemented with 10% fetal bovine serum, 100 IU/mL penicillin, 100 μg/mL streptomycin, and 400 μg/mL G418 (Life Technologies) in a humidified atmosphere of 5% CO2 in air at 37° C. To activate Raf-1 in BON-raf cells, 1 μmol/L h-estradiol (Sigma) was added to the medium. An equivalent amount of ethanol, solvent for the β-estradiol, was used to treat control cells.

ZM336372 Treatment

H727 and BON cells were plated at 50% to 60% confluence in 100-mm cell culture dishes and incubated overnight. Cells were treated with ZM336372 (BioMol, Plymouth Meeting, Pa.) in different concentrations for up to 6 days. Of note, treatments were done in serum-containing medium. Furthermore, the DMSO concentration never exceeded 2% in all treatment groups. ZM336372 at 500 μmol/L concentrations were used only in the toxicity experiments. Stock ZM336372 was made at higher concentration to reduce DMSO toxicity.

Western Blot Analysis

Cellular extracts were prepared and quantified by bicinchoninic acid protein assay kit (Pierce, Rockford, Ill.) as previously described (Sippel & Chen, Surgery 2002;132:1035-9). Denatured proteins (30-50 μg) from each sample underwent electrophoresis on a SDS polyacrylamide gel and transferred to a nitrocellulose membranes (Schleicher and Schuell, Keene, N.H.). Membranes were blocked for 1 hour in milk solution (1×PBS, 5% nonfat dry milk, 0.05% Tween 20) and incubated at 4° C. overnight with primary antibodies. The following primary antibody dilutions were used: phospho-ERK1/2 (1:1,000), phospho-MEK (1:1,000), phospho-Raf-1 (Ser338; 1:1,000), p21 and p18, (1:1,000) (Cell Signaling Technology, Beverly, Mass.), Chromogranin A (1:2,000; Zymed Laboratories, San Francisco, Calif.), MASH-1 (1:1000, BD Pharmingen, San Diego, Calif.), and G3PDH (1:10,000; Trevigen, Gaithersburg, Md.). After primary antibody incubation, membranes were washed 3×5 minutes in PBS-T wash buffer (1×PBS and 0.05% Tween 20). Then the membranes were incubated with either 1:2,000 dilution of goat anti-rabbit or goat anti-mouse secondary antibody (Cell Signaling Technology) depending on the source of the primary antibody for 1 hour at room temperature. Membranes were washed 3×5 minutes in PBS-T wash buffer and developed by Immunstar horseradish peroxidase (Bio-Rad Laboratories, Hercules, Calif.) according to the manufacturer's directions. For the detection of hASH1 protein, membranes were developed with SuperSignal West Femto chemiluminescence reagent (Pierce).

Drug Toxicity Assay

Briefly, cells were trypsinized and plated in a 6-well plate at 1 to 2×10⁶ in triplicate and allowed to adhere overnight. Then the media was replaced with fresh media containing various concentrations (31.25, 62.5, 125, 250, and 500 μmol/L) of ZM336372 and incubated for up to 3 days. As a control, DMSO was added. After incubation, the medium was removed and cells were trypsinized and added to removed medium. Cells were incubated on ice and 2.5 Ag/mL propidium iodide (Sigma) was added 5 minutes before flow cytometry (17). Data was acquired using a FACSCalibur bench top flow cytometer (Becton Dickinson, San Jose, Calif.) using CellQuest acquisition and analysis software.

Cytotoxicity Assay

Cells (H727 and BON) were harvested by trypsinization and plated at a cell density of 3,000 cells per well of each microtiter plate. Cells were grown for 4 hours at 37° C. to allow cell attachment to occur before compound addition. Doxorubicin (control) and ZM336372 were dissolved in DMSO. The final concentration of DMSO in all wells was 2%. Data was compared with the effect of 2% DMSO. Treatments were done in duplicate. Cells were incubated with the test compounds for 72 hours before reading the assay. Then processing and calculation were done according to manufacturer's directions for Cell Titer Glo Assay (Promega, Madison, Wis.).

Cell Proliferation Assay

Proliferation of H727 and BON cells after treatment with ZM336372 was measured using a 3,4-(4,5-dimethylthiazol-2-yl)-2,5-diphenyltetrazolium bromide (Sigma). Cells were trypsinized and plated in triplicate to 24-well plates and allowed to adhere overnight. Then, cells were treated with either 100 μmol/L ZM336372 or DMSO (2%) and incubated. Media were changed every 2 days with new treatment. At each time point, cell growth rates were analyzed after the addition of 3,4-(4,5-dimethylthiazol-2-yl)-2,5-diphenyltetrazolium bromide reagent to the cultured cells following manufacturer's instructions. Absorbance was determined using spectrophotometer at a wavelength of 540 nm.

Results

Raf-1 Activation Results in Reduction of hASH1 in Carcinoid Tumor Cells

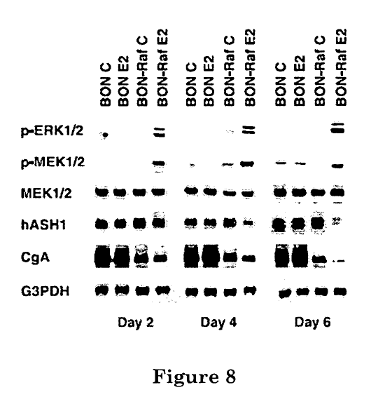

Raf-1 activation in carcinoid tumor cells has been previously shown to result in activation of the Raf-1 pathway mediators MEK1/2 and ERK1/2 and reduction in chromogranin A. Untreated, native carcinoid tumor cells (BON C) have little to no phosphorylation of MEK1/2 or ERK1/2 at baseline (FIG. 8). Furthermore, native carcinoid cells have high levels of chromogranin A and hASH1. Activation of Raf-1 in gastrointestinal carcinoid BON-raf cells by estradiol treatment leads to activation of MEK1/2 and ERK1/2 as well as reduction in chromogranin A compared with controls (FIG. 8). Because hASH1 is highly expressed in neuroendocrine tumor cells and has been shown to mediate the neuroendocrine phenotype, the decrease in neuroendocrine markers induced by Raf-1 pathway activation could be due to a decrease in hASH1. Estradiol (E2)-induced activation of Raf-1 in BON-raf tumor cells resulted in significant reductions in hASH1 by Western analysis at days 2, 4, and 6 (FIG. 8), suggesting that Raf-1 may play a role in regulation of this transcription factor in carcinoid tumors.

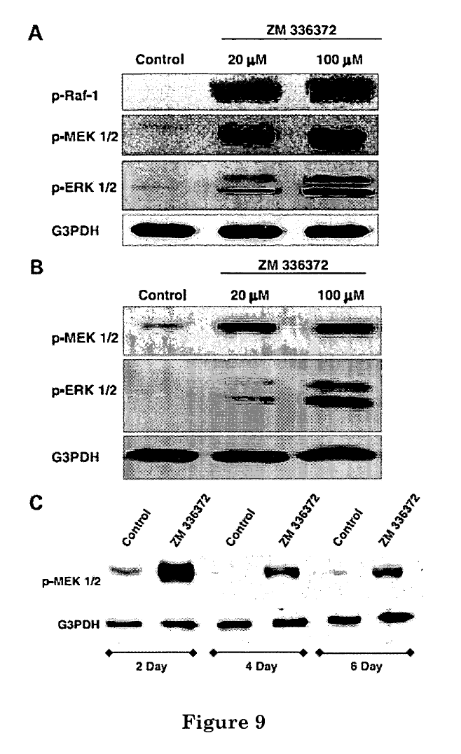

ZM336372 Activates the Raf-1/MEK/ERK System in a Dose-Dependent Manner

Whereas ZM336372 has been shown to activate Raf-1 ex vivo, there are little data to illustrate that this leads to phosphorylation of downstream mediators such as ERK1/2 and MEK1/2 in vitro. Therefore, Western analysis was used to show that Raf-1/MEK/ERK pathway activation occurs in response to treatment with ZM336372 in carcinoid tumor cells. In control pulmonary (H727) and gastrointestinal (BON) carcinoid tumor cells, there is little phosphorylation of Raf-1, MEK1/2, or ERK1/2. At 2 days, treatment with 20 and 100 μmol/L ZM336372 led to activation of MEK1/2 and ERK1/2 in native H727 cells compared with control (DMSO) treatments as evidenced by protein phosphorylation (FIG. 9A). Furthermore, Raf-1 is also phosphorylated at Ser338 by addition of ZM336372, indicating that this system is activated at least at the level of Raf-1. Similarly, in BON tumor cells, there is strong activation of ERK1/2 and MEK1/2 with increasing doses of ZM336372 (FIG. 9B). Moreover, activation of the Raf-1/MEK/ERK system is sustained, as exemplified by MEK1/2 phosphorylation in H727 cells at days 4 and 6 with the addition of 100 μmol/L ZM336372 (FIG. 9C), showing prolonged action of this compound.

ZM336372 Reduces Neuroendocrine Hormone Production in Carcinoid Tumor Cells

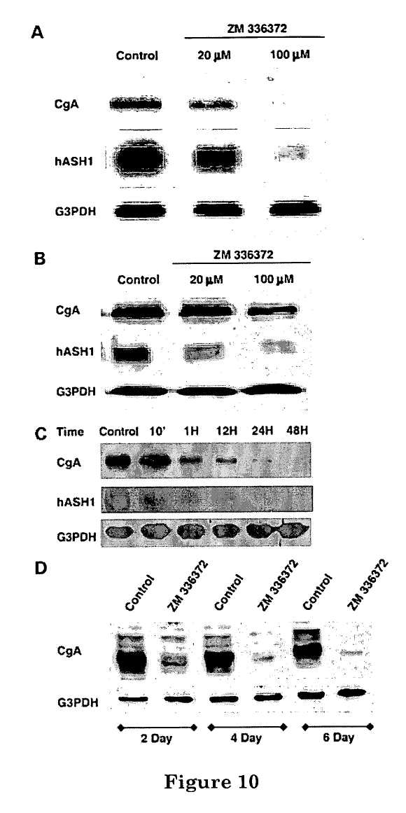

Changes in chromogranin A levels are known to be concordant with alterations in other neuroendocrine hormones such as histamine and serotonin (Sippel & Chen, Surgery 2002;132:1035-9). Additionally, hASH1 is expressed in most neuroendocrine tumors and correlates with neuroendocrine hormone levels as previously described. Therefore, to determine if ZM336372 can reduce chromogranin A and hASH1, pulmonary H727 and gastrointestinal BON carcinoid cells were treated with ZM336372. Untreated, native H727 and BON cells have high levels of chromogranin A and hASH1. However, treatment of H727 and BON with ZM336372 caused reduction in both chromogranin A and hASH1 as shown by Western analysis (FIGS. 10A and B). Furthermore, to determine when chromogranin A depletion occurs, H727 cells were treated with ZM336372 at different time intervals. Earliest detection of reduction of chromogranin A with treatment occurred at 1 hour; however, there was temporal reduction in chromogranin A with the greatest comparative loss at 48 hours (FIG. 10C). These results also correlate with hASH1 reduction at similar time points. Moreover, to determine if ZM336372 had a persistent effect with regard to neuroendocrine hormone depletion, Western analysis of H727 cells treated with the drug or carrier control at days 2, 4, and 6 was carried out. Treatment with ZM336372 had a lasting effect as chromogranin A depletion was maintained to 6 days with treatment in carcinoid cells (FIG. 10D).

ZM336372 Suppresses Cell Proliferation and Induces Cell Cycle Inhibitors in Carcinoid Tumor Cells

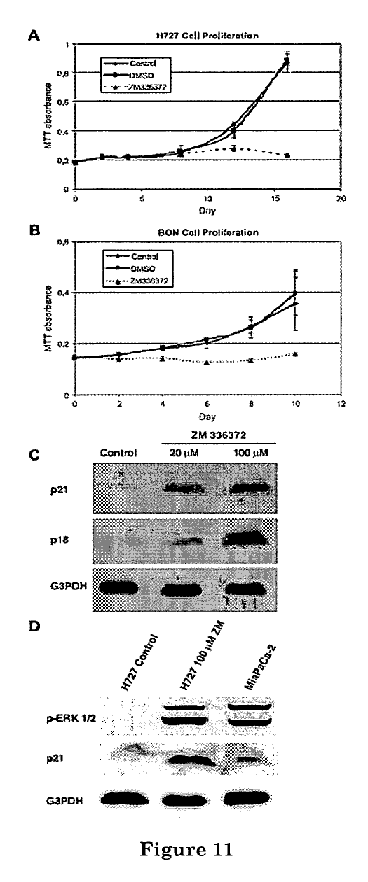

Raf-1 activation results in growth inhibition in many cell types, as well as cell senescence in others (Ravi et al., J Clin Invest 1998;101: 153-9; Park et al., Mol Cell Biol 2003;23:543-54; Woods et al., Mol Cell Biol 1997;17: 5598-611). Initially, when treating H727 cells plated at 50% confluence with ZM336372, it was noticed that the cells never reached confluence as nontreatment cells would at days 2, 4, or 6 (data not shown). This observation was substantiated by 3,4-(4,5-dimethylthiazol-2-yl)-2,5-diphenyltetrazolium bromide growth assay. H727 cells treated with ZM336372 were growth suppressed, whereas control treatments had significantly more growth by day 6, continuing up to 16 days (FIG. 11A). A similar response was also seen in BON cells as growth suppression occurred as early as day 4 and was maintained out to day 10 (FIG. 11B). All experiments were done in serum-containing medium including control and ZM336372 treatments. However, growth arrest was only seen in ZM336372-treated cells.

ZM336372 also inhibited growth of other neuroendocrine tumor cell lines including medullary thyroid cancer cells (data not shown). It is possible ZM336372 may have more pronounced growth inhibition in the absence of serum.

The ability of ZM336372 to suppress cellular proliferation through Raf-1 activation seems specific to neuroendocrine tumor cells. Raf-1 activation has been shown to induce expression of cell cycle inhibitors of separate families, including p21 and those of the INK family, such as p18 (Woods et al., Mol Cell Biol 1997;17: 5598-611). As shown in FIG. 11C, carcinoid tumor cells have minimal levels of p21 and p18 at baseline, but treatment with ZM336372 induced p21 and p18 at 2 days compared with controls. In contrast, in human pancreatic cancer cell lines (e.g. MiaPaCa2) that have constitutive activation of the ras/Raf-1/MEK/ERK1/2 pathway (as evidenced by the presence of high levels of phosphorylated, active ERK1/2), there was no up-regulation of p21 (FIG. 11D).

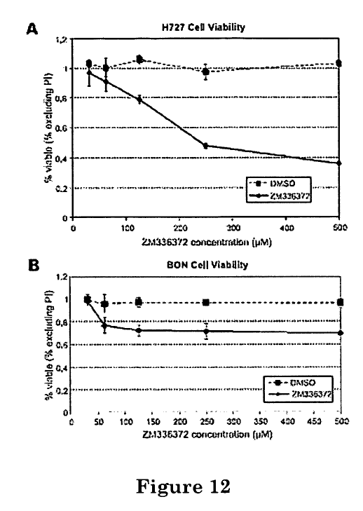

Propidium iodide exclusion assay was performed to assess direct cytotoxicity of ZM336372. As seen in FIG. 12, at high concentrations of the drug (500 Amol/L), 40% to 50% of H727 cells remain viable at 2 days with treatment compared with native H727 cells. Furthermore, at concentrations used in this article (20-100 μmol/L), cytolysis is <20%. Moreover, in BON cells, ˜70% of cells remain viable by propidium iodide exclusion at 2 days from 63 to 500 μmol/L ZM336372. As a control, H727 and BON cells were treated with doxorubicin, an agent known to cause significant cytotoxicity to tumor cells, to validate the assay. IC₅₀ values were obtained with doxorubicin of 1.1 and 1.4 μmol/L in H727 and BON tumor cells, respectively. However, ZM336372 addition to H727 and BON cells in concentrations ranging from 0 to 200 μmol/L did not produce enough cellular cytotoxicity to estimate an IC50 (data not shown).

### Example 10

### RNA Interference (RNAi) Against GSK3B Results in GSK3B Protein Expression

RNAi has been successfully used to reduce protein expression in the Raf-1 signaling pathway, such as hASH1 and AKT. For example, transfection of siRNA against AKT results in a reduction in AKT protein by Western blot in MTC TT cells. siRNA against AKT and hASH1 also blocked protein expression for at least 5 days (data not shown). The degree of inhibition can be effectively controlled by titrating the dose of siRNA.

siRNA Design Two siRNAs are used against GSK3B which have recently been shown to completely inhibit GSK3B (Jiang et al., Cell 2005; 120:123-135; Yu et al., Molecular Therapy 2003; 7:228-236.). The sequences of the two siRNAs are GSK3BHP1 (5′-GAUCUGGAGCUCUCGGUUCU-3′) (SEQ ID NO: 1), and GSK3BHP2 (5′-GUGUUGCUGAGUGGCACUCA-3′) (SEQ ID NO: 2)

As negative controls, siRNAs with every 3rd base mutated, GSK3BHP1-MUT and GSK3BHP2-MUT, are synthesized.

Treatment of Cell Lines GSK3BHP1, GSK3BHP2, and control mutated siRNAs are transfected into pulmonary carcinoid H727, medullary thyroid cancer TT, and pancreatic carcinoid BON cells using Oligofectamine plus (Mirus) and with dosages ranging between 4-8 nM, appropriate for each cell line. NE tumor cells are exposed to the siRNAs for up to 5 days. Cellular lysates are harvested at 24, 48, and 96 hours.

GSK3B and NE marker expression Cellular extracts are analyzed using western blots with GSK3B, hASH1, chromogranin A, synaptophysin, and neuron-specific enolase antibodies, as described above. Serotonin and calcitonin secretion are also assessed using an ELISA assay as described above. The in vitro conditions that results a significant reduction in GSK3B protein (non-phosphorylated, active protein) are optiminzed. Levels of phosphorylated GSK3B, which is normally minimal at baseline, are also measured.

Cellular proliferation After optimizing the concentrations of siRNAs to block GSK3B, suppression of cellular growth due to inhibition GSK3B is determined. BON, TT, and H727 cells are treated with varying dosages of siRNAs. Cellular proliferation is analyzed by a colorimetric assay using 3-(4,5-dimethylthiazol-2-yl)-2,5-diphenyltetrazolium bromide (MTT) (Sigma) over a 5-day time period. In addition, standard viable cell count growth curves are determined.

Treatment with GSK3B siRNAs results in a reduction in NE marker expression and cellular growth. To improve siRNA stability, stable NE cell lines with inducible GSK3B siRNA constructs utilizing a doxycycline inducible siRNA vector (available from Genscript corporation) is used. Tetracycline inducible systems may also be used, and multiple NE cells have been established with inducible genes which do not have leaky background expression in the absence of tetracycline and strong dose-dependent induction. Tetracycline-inducible vectors are known and readily available, e.g. the Tet-on vectors commercially from Clonetech, to those skilled in the art.

### Example 11

### Inhibiting GSK3B Function Using a Dominant Negative Form of Human GSK3B

An alternative strategy to treatment with GSK3B siRNAs is the use of dominant-negative constructs. A well-characterized dominant negative form of human GSK3B is available from J. Sadoshima (Penn State Univ.).

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. Furthermore, the teachings and disclosures of all references cited herein are expressly incorporated in their entireties by reference.

What is claimed is:

1. A method for treating a neuroendocrine (NE) tumor, or for inhibiting or reducing symptoms of NE tumor in a patient, comprising administering to the patient a therapeutically effective amount of a pharmaceutical composition which comprises a pharmaceutically acceptable amount of a GSK-3 specific inhibitor that is sufficient to block or inhibit activity of GSK-3 in the patient.

2. The method of claim 1, wherein the patient is a mammal.

3. The method of claim 2, wherein the mammal is human.

4. The method according to claim 1, wherein the GSK3 is GSK3β.

5. The method of claim 4, wherein the GSK-3β inhibitor comprises a substance selected from the group consisting of Li+, SB216763, SB415286, indirubins, Paullones, Hymenialdisine, Azakenpaullone, Thienyl and phenyl α-halomethyl ketones, CHR 99021, AR-A014418, Bis-7-azaindolylmaleimides, CHR 98023, CHR-98014, and ZM336372, or a pharmaceutically acceptable salt or derivative thereof.

6. The method of claim 1, wherein the GSK-3 inhibitor comprises an anti-GSK-3 antibody, or a polynucleotide molecule comprising a sequence that is antisense to a nucleic acid encoding a GSK-3, or a small interfering RNA (siRNA) based on a nucleic acid encoding a GSK-3.

7. The method according to claim 6, wherein the GSK3 is GSK3β.

8. The method according to claim 6, wherein the antisense nucleic acid sequence is antisense to SEQ ID NO:3.

9. The method according to claim 6, wherein the siRNA is based on a genomic sequence encoding GSK-3β, or a cDNA sequence encoding GSK3β.

10. The method of claim 1, wherein blocking or inhibiting the GSK-3 activity in the patient results in reducing formation of chromogranin A (CgA) or human achaete-scute homolog-1 (HASH1) in NE tumor cells.

11. The method of claim 1, wherein the NE tumor is selected from the group consisting of carcinoids, islet cell tumors, and medullary thyroid cancers.

12. A pharmaceutical composition for treating a neuroendocrine (NE) tumor, or for inhibiting or reducing symptoms of NE tumor in a patient, comprising a therapeutically effective amount of a pharmaceutically acceptable amount of a GSK-3 specific inhibitor that is sufficient to block or inhibit activity of GSK-3 in the patient, and a pharmaceutically acceptable excipient.

13. The pharmaceutical composition of claim 12, wherein the patient is a mammal.

14. The pharmaceutical composition of claim 13, wherein the mammal is human.

15. The pharmaceutical composition according to claim 12, wherein the GSK3 is GSK3β.

16. The pharmaceutical composition of claim 15, wherein the GSK-3β inhibitor comprises a substance selected from the group consisting of Li+, SB216763, SB415286, indirubins, Paullones, Hymenialdisine, Azakenpaullone, Thienyl and phenyl α-halomethyl ketones, CHR 99021, AR-A014418, Bis-7-azaindolylmaleimides, CHR 98023, CHR-98014, and ZM336372, or a pharmaceutically acceptable salt or derivative thereof.

17. The pharmaceutical composition of claim 15, wherein the GSK-3β inhibitor comprises at least one of Li+, SB216763, or ZM336372, or a pharmaceutically acceptable salt thereof.

18. The pharmaceutical composition of claim 12, wherein the GSK-3 inhibitor comprises an anti-GSK-3 antibody, or a polynucleotide molecule comprising a sequence that is antisense to a nucleic acid encoding a GSK-3, or a small interfering RNA (siRNA) based on a nucleic acid encoding a GSK-3.

19. The pharmaceutical composition according to claim 18, wherein the GSK3 is GSK3β.

20. The pharmaceutical composition according to claim 19, wherein the antisense nucleic acid sequence is antisense to SEQ ID NO:3.

21. The pharmaceutical composition according to claim 19, wherein the siRNA is based on a genomic sequence encoding GSK-3β, or a cDNA sequence encoding GSK3β.

22. The pharmaceutical composition of claim 12, wherein blocking or inhibiting the GSK-3 activity in the patient results in reducing formation of chromogranin A (CgA) or human achaete-scute homolog-1 (hASH1) in NE tumor cells.

23. The pharmaceutical composition of claim 12, wherein the NE tumor is selected from the group consisting of carcinoids, islet cell tumors, and medullary thyroid cancers.

24. The pharmaceutical composition of claim 1, wherein the GSK-3β inhibitor comprises lithium or a pharmaceutically acceptable salt thereof.

25. A kit comprising the pharmaceutical composition according to claim 12, and an instructional material regarding the use thereof to treat an NE tumor or reducing a symptom thereof.

26. The kit of claim 25, further comprising a delivery device for delivering the composition to a patient.

27. The kit of claim 25, wherein the GSK3β inhibitor comprises Li+.
